(19) United States
(12) Patent Application Publication
(10) Pub. No.: US 2024/0053326 A1
(43) Pub. Date: Feb. 15, 2024

(54) TESTING COLON CANCER DRUGS FOR IMMUNOMODULATORY EFFECTS

(71) Applicant: BROWN UNIVERSITY, Providence, RI (US)

(72) Inventors: Wafik S. El-Deiry, Barrington, RI (US); Kelsey E. Huntington, Providence, RI (US); Anna Louie, Providence, RI (US)

(21) Appl. No.: 18/230,872

(22) Filed: Aug. 7, 2023

Related U.S. Application Data
(60) Provisional application No. 63/395,849, filed on Aug. 7, 2022.

(51) Int. Cl. G01N 33/50

(52) U.S. Cl. CPC G01N 33/5041; G01N 2800/52; G01N 2333/475; G01N 2333/521

(57) ABSTRACT

A method of treating a subject having colon cancer with a small molecule drug for treating colon cancer is described. The method includes determining the effect of the small molecule drug on the release of a plurality of immunomodulating factors by colon cancer cells; and treating the subject with a therapeutically effect amount of the drug if the effect of the drug on the release of immunomodulating factors indicates that the drug promotes anti-tumor immunity, or selecting a different drug for treating colon cancer if the effect of the drug on immunomodulating factor release indicates that the drug does not promote anti-tumor immunity. Methods of evaluating ongoing treatment using the method are also described.

### CROSS-REFERENCE TO RELATED APPLICATIONS

This application claims priority to U.S. Provisional Application Ser. No. 63/395,849, filed on Aug. 7, 2022, which is hereby incorporated by reference in its entirety.

### STATEMENT REGARDING FEDERALLY SPONSORED RESEARCH

This invention was made with government support under Grant Number CA173453 awarded by the National Institutes of Health. The Government has certain rights in this invention.

### BACKGROUND

Cytokines, chemokines, and growth factors are all molecular messengers of the immune system that impact tumor behavior and host response. Cytokines are either secreted or membrane-bound proteins that regulate cellular signaling and can be categorized as pro- or anti-inflammatory. Chemokines are proteins that mediate chemotaxis in nearby cells and play an important role in the recruitment of either immunosuppressive or immunostimulatory cell types to the tumor microenvironment. Differential expression of chemokines regulates the selective migration of myriad cell types. Turner M D, et al., Biochim Biophys Acta. 2014; 1843:2563-82. Growth factors are usually secreted proteins or steroid hormones that promote cell proliferation and differentiation. Each of these categories of immunomodulating agents are produced by both tumor and immune cells, among other cell types, and can impact therapeutic response. Berraondo P, et al., Br J Cancer. 2019; 120:6-15.

The characterization of these soluble mediators as biomarkers of both diagnosis and prognosis is a rapidly evolving topic in cancer research and clinical oncology. Biomarkers have been correlated with clinical outcome in several different tumor types including colorectal cancer (CRC) Yamaguchi M, et al., PLoS One. 2019; 14:e0213602. In CRC, differentially expressed plasma or serum cytokines represent potential biomarkers for diagnosis and prognosis. Yamaguchi et al. found that the levels of cytokines in plasma varied significantly between patients with CRC and control subjects. Yamaguchi M, et al., PLoS One. 2019; 14:e0213602. However, cytokine signaling is highly pleotropic with one cytokine producing diverse and sometimes opposing effects depending on the signaling context. Berraondo P, et al., Br J Cancer. 2019; 120:6-15. Moreover, cytokine signaling is characterized by a high degree of redundancy where discrete cytokines produce the same functional effects. Ozaki K and Leonard W J, J Biol Chem. 2002; 277:29355-58. The combination of pleotropic and redundant outcomes in response to a particular cytokine makes therapeutic manipulation challenging. Furthermore, there exists a degree of heterogeneity in the prognostic value of cytokines, with some showing opposing correlations in response to therapy across multiple tumor types. Park J W, et al., Br J Cancer. 2020; 123:610-18. Accordingly, what is needed is a method of obtaining cytokine profiles from tumor samples that can be used to more accurately predict the therapeutic response to immunotherapies such as immune checkpoint blockade.

### SUMMARY

Inflammatory cytokines, chemokines, and growth factors are molecular messengers that circulate and have the capability to modify the tumor microenvironment and impact therapeutic response. The characterization of soluble mediators as biomarkers for diagnosis and prognosis is of interest in oncology. We utilize the cytokinome to characterize the response of colorectal tumor cell lines to selected small-molecules in oncology as a proof-of-concept dataset with immunomodulatory analyte heat map rankings for drug and cell line combinations. We observed overall trends in drug class effects with MEK-, BRAF-, PARP-inhibitors, and Imipridones in cytokine, chemokine, and growth factor responses that may help guide therapy selection. MEK-inhibitor treatment downregulated analytes VEGF, CXCL9/MIG, and IL-8/CXCL8 and upregulated CXCL14/BRAK, Prolactin, and CCL5/RANTES. BRAF-inhibitor treatment downregulated VEGF and IL-8/CXCL8, while increasing soluble TRAIL-R2. Treatment with PARP-inhibitors decreased CXCL9/MIG, IL-8/CXCL8, CCL3/MIP-1 alpha, VEGF, and CXCL14/BRAK, while treatment increased soluble TRAIL-R2 and prolactin. Treatment with Imipridones decreased CCL3/MIP-1 alpha, VEGF, CXCL14/BRAK, IL-8/CXCL8, and Prolactin and increased CXCL5/ENA-78. We also observed differential responses to therapeutics depending on the mutational profile of the cell line. In the future, a similar but larger dataset may be utilized in the clinic to aid in the prediction of patient response to immunomodulatory therapies based on tumor genotype. Accordingly, the present invention provides a tool to help predict which patients would respond best to immunotherapies such as immune checkpoint blockade.

### BRIEF DESCRIPTION OF THE FIGURES

FIG. 1 provides graphs showing the IC-50 curves for small molecules analyzed in HCT-116, HT-29, and KM12C. Cell viability curves post 72-hour treatment were graphed in GraphPad and used to determine IC-50 values.

FIG. 2 provides a scheme showing the cell culture supernatant cytokinome analysis workflow. (A) Small-molecules in oncology were selected to provide varied mechanisms of action. (B) Three colon cancer cell lines (HCT-116, HT-29, and KM12C) were selected based on differing mutational backgrounds in key tumor suppressor genes. (C) Cytokinome analysis was performed on cell culture supernatant samples after 48 hours of treatment using a Luminex 200. (D) Biomarker-based predictions about diagnosis, prognosis, and response to therapy were made based on heat maps generated from linear regression analysis of the data.

FIG. 3 provides an immune synergy heat map showing cell line changes in cytokine, chemokine, and growth factor profiles in response to therapeutic treatment. Cytokines, chemokine, and growth factors are grouped into one of two categories: (1) analytes that are correlated with immunosuppression or unfavorable prognosis or (2) analytes that are correlated with immunostimulation or favorable prognosis in the context of CRC. The heat map is based on the slope of the linear regression where green indicates upregulation, yellow indicates no change, and red indicates downregulation post-treatment.

FIGS. 4A and 4B provide heatmaps displaying regression slopes of cytokine profiles for MEK inhibitors. (A) Heat maps based on regression slopes for HCT-116, HT-29, and KM12C after 48-hour treatment of increasing doses of Selumetinib or (B) Trametinib.

FIGS. 5A and 5B provide heatmaps displaying regression slopes of cytokine profiles for BRAF inhibitors. (A) Heat maps based on regression slopes for HCT-116, HT-29, and KM12C after 48-hour treatment of increasing doses of Regorafenib or (B) Vemurafenib.

FIGS. 6A and 6B provide heatmaps displaying regression slopes of cytokine profiles for PARP inhibitors. (A) Heat maps based on regression slopes for HCT-116, HT-29, and KM12C after 48-hour treatment of increasing doses of Olaparib or (B) Rucaparib.

FIGS. 7A-7C provide heatmaps displaying regression slopes of cytokine profiles for imipridones. (A) Heat maps based on regression slopes for HCT-116, HT-29, and KM12C after 48-hour treatment of increasing doses of ONC201, (B) ONC206, or (C) ONC212.

### DETAILED DESCRIPTION OF THE INVENTION

The present invention provides a method of treating a subject having colon cancer with a small molecule drug for treating colon cancer. The method includes determining the effect of the small molecule drug on the release of a plurality of immunomodulating factors by colon cancer cells; and treating the subject with a therapeutically effect amount of the drug if the effect of the drug on the release of immunomodulating factors indicates that the drug promotes anti-tumor immunity, or selecting a different drug for treating colon cancer if the effect of the drug on immunomodulating factor release indicates that the drug does not promote anti-tumor immunity. Use of the method to evaluate ongoing treatment in subjects is also described.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction or differentiation of the undesirable proliferating cells with minimal effects on normal cells. In accordance with the present invention, desired mechanisms of treatment at the cellular level include stimulation of differentiation in cancer and pre-cancer cells.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of cancer growth by a detectable amount.

The term “effective amount” as used herein refers to an amount sufficient to achieve an intended result. An “effective amount” includes an amount that is 100% effective in achieving that result, but also includes amounts that are less effective but still exhibit a significant effect. For example, an effective amount of a compound for reducing toxic side effects is an amount sufficient to reduce, but not necessarily eliminate, those effects.

The term “antibody” as used herein refers to immunoglobulin molecules or other molecules which comprise at least one antigen-binding domain. The term “antibody” as used herein is intended to include whole antibodies, monoclonal antibodies, polyclonal antibodies, chimeric antibodies, humanized antibodies, primatized antibodies, multi-specific antibodies, single chain antibodies, epitope-binding fragments, e.g., Fab, Fab′ and F(ab′)2, Fd, Fvs, single-chain Fvs (scFv), disulfide-linked Fvs (sdFv), fragments comprising either a VL or VH domain, and totally synthetic and recombinant antibodies. The antibodies can be of any type (e.g., IgG, IgE, IgM, IgD, IgA, and IgY), class (e.g., IgG1, IgG2, IgG3, IgG4, IgA1 and IgA2) or subclass of immunoglobulin molecule.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

### Methods of Colon Cancer Treatment

A method of treating a subject having colon cancer with a small molecule drug for treating colon cancer, comprising determining the effect of the small molecule drug on the release of a plurality of immunomodulating factors by colon cancer cells; and treating the subject with a therapeutically effect amount of the drug if the effect of the drug on the release of immunomodulating factors indicates that the drug promotes anti-tumor immunity, or selecting a different drug for treating colon cancer if the effect of the drug on immunomodulating factor release indicates that the drug does not promote anti-tumor immunity. In some embodiments, if a different drug is selected, it is a drug that uses a different mechanism of action than the drug that was tested for its effect on the release of immunomodulating factors (i.e., the initial drug).

The method includes the step of determining the effect of the small molecule drug on the release of a plurality of immunomodulating factors by colon cancer cells. Immunomodulating factors are in vivo compounds that either stimulate or suppress immune function. Examples of immunomodulating factors include cytokines, chemokines, and growth factors. Some compounds can be characterized as two or more of these; for example, some compounds are both cytokines and growth factors.

In some embodiments, the immunomodulating factors comprise cytokines. Cytokines (CK) are low-molecular-weight soluble proteins produced by various cells induced by immunogens, mitogens or other stimulators, and have many functions such as regulating innate and adaptive immunity, hematopoiesis, cell growth, and repair damaged tissues. Cytokines can be divided into interleukins, interferons, tumor necrosis factor superfamily, colony stimulating factors, chemokines, growth factors and the like. In some embodiments, the cytokines are selected from the group consisting of IL-7, IL-2, CRP, TRAIL R3, TRAIL R2, Prolactin, and PD-L1/B7-H1.

In some embodiments, the immunomodulating factors comprise chemokines. Chemokines are a large family of relatively small (typically 8-14 kDa) chemoattractant proteins that play a central role in controlling leukocyte migration during development, homeostasis, and inflammation. In some embodiments, the chemokines are selected from the group consisting of CXCL14/BRAK, CXCL10/IP-10, CCL5/RANTES, CXCL11/I-TAC, CXCL9/MIG, CCl3/MIP-1 alpha, CC11/Eotaxin, CXCL5/ENA-78, CXCL13/BLC/BCA-1, and IL-8/CXCL8.

In some embodiments, the immunomodulating factors comprise growth factors. Growth factors are a class of peptides that bind to specific, high-affinity cell membrane receptors and regulate multiple effects such as cell growth and other cellular functions. They are secreted by a variety of cells, acting on specific target cells and regulating cell division, matrix synthesis and tissue differentiation. In some embodiments, the growth factors are selected from the group consisting of IL-18, IFN-beta, IFN-alpha, TRANCE, GM-CSF, M-CSF, IL-10, IL-6, VEGF, and TNF-alpha.

The present invention includes the step of treating cancer in a subject. Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

The invention is directed to methods of treating colon cancer. Colon cancer is also known as colorectal cancer, bowel cancer, and rectal cancer. Colorectal cancer generally originates from the epithelial cells lining the colon or rectum of the gastrointestinal tract, most frequently as a result of genetic mutations in the Wnt signaling pathway that increases signaling activity. The signs and symptoms of colorectal cancer depend on the location of the tumor in the bowel, and are known to those skilled in the art.

A wide variety of methods are known for colon cancer treatment, which vary depending on the type and stage of the cancer being treated. These methods can be used in addition to the administration of a therapeutically effective amount of a small molecule drug (i.e., chemotherapy). Methods of cancer treatment include radiation therapy, radiofrequency ablation, cryoablation, thermal ablation, electroporation, alcohol ablation, high intensity focused ultrasound, chimeric antigen receptor (CAR) T-Cell therapy, administration of monoclonal antibodies, immune checkpoint blockade, and administration of immunotoxins. Cancer treatment can be used for both prophylactic and therapeutic treatment.

Colon cancer therapy can, for example, be provided prophylactically to a subject prior to the development of cancer. Prophylactic administration, also referred to as prevention, is effective to decrease the likelihood that cancer will develop in the subject. The subject in need of prophylactic treatment may be an individual who has or is suspected of having a colon cancer. In some of variations, the human is at risk of developing colon cancer (e.g., a human who is genetically or otherwise predisposed to developing colon cancer) and who has or has not been diagnosed with colon cancer. As used herein, an “at risk” subject is a subject who is at risk of developing colon cancer. The subject may or may not have detectable disease, and may or may not have displayed detectable disease prior to the treatment methods described herein. An at-risk subject may have one or more so-called risk factors, which are measurable parameters that correlate with development of colon cancer, such as described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, inflammatory bowel disease, and environmental exposure. In some embodiments, a human at risk for colon cancer includes, for example, a human whose relatives have experienced this disease, and those whose risk is determined by analysis of genetic or biochemical markers. Prior history of having colon cancer may also be a risk factor for instances of cancer recurrence. In some embodiments, provided herein is a method for treating a human who exhibits one or more symptoms associated with colon cancer.

The effectiveness of cancer treatment may be measured by evaluating a reduction in tumor load or decrease in tumor growth in a subject in response to the administration of the modified immune suppressor cells. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

A wide variety of different types of small molecule drugs are known for use in treating colon cancer. Small molecule drugs are organic compounds having a relatively low molecular weight. Larger structures such as polynucleotides and proteins are not considered small molecules, although their components (nucleotides and amino acids) can be. Small molecule drugs typically have a molecular weight of <1000 daltons. Preferably the small molecule drugs have he ability to pass through cell membranes to reach intracellular targets. In some embodiments, various different concentration values for the small molecules are used when determining their effect on immunomodulating factors.

Embodiments of the invention can make use of different types of small molecule drugs for treating colon cancer. In some embodiments, the drug is a tyrosine kinase inhibitor. Examples of tyrosine kinase inhibitors include Larotrectinib, regorafenib, and dasatinib. In further embodiments, the drug is an imipridone compound. The angular structure of the imipridone scaffold has been identified. Wagner et al., Oncotarget, 5 (24), 12728 (2014) and a variety of imipridone compounds have been identified. Prabhu et al., Neoplasia, 22, 12, 725 (2020). Examples of imipridone compounds that can be used for treating colon cancer include ONC201, ONC206, and ONC212. The structure of ONC201 is shown below:

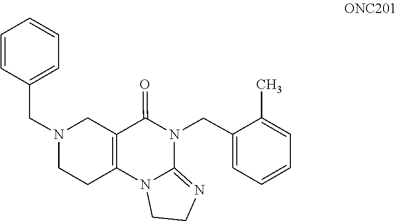 [SMILES: Cc1ccccc1CN1C(=O)C2=C(CCN(Cc3ccccc3)C2)N2CCN=C12]

Other small molecule drugs that can be used for treating colon cancer can be selected from the group consisting of MEK inhibitors (e.g., trametinib, selumetinib), RET inhibitors (e.g., selpercatinib), BRAF inhibitors (e.g., vemurafenib, dabrafenib), PARP inhibitors (e.g., rucaparib, Olaparib), P13K inhibitors (e.g., duvelisib), and GSK-3 inhibitors (e.g., 9-ING-41).

In some embodiments, the effect of the small molecule drug on the release of a plurality of immunomodulating factors is evaluated in a plurality of different types of colon cancer cells, which can be referred to herein as test cells. Preferably, the different types of colon cancer cells are colon cancer cells having diverse mutational backgrounds. Mutational background can affect tumor suppressor or oncogenic drivers expressed by the cells. For example, different types of colon cancer cells can express different levels of TP53, HRAS, NRAS, KRAS, BRAF, PIK3CA, PTEN, APC, TRK, CTNNB1, ACVR2A, BRCA2, and TGFBR2, and can exhibit different levels of microsatellite stability. The use of tumor cells having diverse mutational backgrounds increases the ability to predict the effect of the small molecular colon cancer drug on immunomodulating factor release by cancer cells in a subject. In some embodiments, the test cells include colon cancer cells obtained from the subject.

In some embodiments, the levels of the immunomodulating factors are determined using an immunoassay. As used herein, the term “immunoassay” refers to an assay in which an antibody specifically binds to a protein including an antigen recognized by the capture antibody to provide for the detection and/or quantitation of the protein. An “immunoassay” can use a particular antibody to detect, isolate, target, and/or quantify the antibody that specifically binds to the capture antibody. One example of an “immunoassay” includes a capture antibody that contains one or more antigens to detect, isolate, and/or quantify one or more proteins in a sample. Immunoassays can be run using a variety of different formats, including competitive homogenous immunoassays, heterogeneous immunoassays, one-site non-competitive immunoassays, and two-site non-competitive immunoassays. In some embodiments, the immunoassay is an enzyme-linked immunosorbent assays (ELISA).

Antibodies are designed for specific binding, as a result of the affinity of complementary determining region of the antibody for the epitope of the biological analyte. An antibody “specifically binds” when the antibody preferentially binds a target structure, or subunit thereof, but binds to a substantially lesser degree or does not bind to a biological molecule that is not a target structure.

In some embodiments, the levels of the immunomodulating factors are determined using a multiplex kit. A multiplex kit, as used herein, is an immunoassay that includes the capacity to recognize a large number, or plurality, of immunomodulating factors using a single kit. For example, a multiplex kit can provide the capacity to recognize at least 10, at least 50, or at least 100 different immunomodulating factors. An example of a commercially available multiplex kit are Luminex® assays provided by R&D Systems™

### Methods of Evaluating Treatment

A method of evaluating treatment a subject having colon cancer being treated with a therapeutically effective amount of a small molecule drug for treating colon cancer, comprising determining the effect of the small molecule drug on the release of a plurality of immunomodulating factors by colon cancer cells; and continuing to treat the subject if the effect of the drug on the release of immunomodulating factors indicates that the drug promotes anti-tumor immunity, or changing treatment to a different drug for treating colon cancer if the effect of the drug on immunomodulating factor release indicates that the drug does not promote anti-tumor immunity. In some embodiments, if a different drug is selected, it is a drug that uses a different mechanism of action than the drug that was tested for its effect on the release of immunomodulating factors (i.e., the initial drug).

The method includes the step of determining the effect of the small molecule drug on the release of a plurality of immunomodulating factors by colon cancer cells. The method includes the use of any of the small molecule drugs or immunomodulating factors described herein. In some embodiments, the immunomodulating factors comprise cytokines. In further embodiments, the immunomodulating factors comprise chemokines. In yet further embodiments, the immunomodulating factors comprise growth factors.

In some embodiments, the effect of the small molecule drug on the release of a plurality of immunomodulating factors is evaluated in a plurality of different types of colon cancer cells. In further embodiments, the levels of the immunomodulating factors are determined using an immunoassay. In yet further embodiments, the levels of the immunomodulating factors are determined using a multiplex kit.

### Administration and Formulation

The present invention includes pharmaceutical compositions that include a small molecule colon cancer drug as an active ingredient, which can also include a pharmaceutically acceptable carrier or carriers, in combination with the active ingredient.

The colon cancer drugs can be administered as pharmaceutically acceptable salts. Pharmaceutically acceptable salt refers to the relatively non-toxic, inorganic and organic acid addition salts of the compounds. These salts can be prepared in situ during the final isolation and purification of the compounds, or by separately reacting purified compounds with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions include small molecule drugs together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The small molecule colon cancer drug can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The compounds are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing the compounds, or as a solution or suspension in an aqueous liquor or non-aqueous liquid such as a syrup, an elixir, an emulsion, or a draught. Such compositions and preparations typically contain at least about 0.1 wt-% of the active agent. The amount of the compound is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

An example has been included to more clearly describe particular embodiments of the invention. However, there are a wide variety of other embodiments within the scope of the present invention, which should not be limited to the particular example provided herein.

### Example

### A High-Throughput Customized Cytokinome Screen of Colon Cancer Cell Responses to Small-Molecule Oncology Drugs

Cytokinome Profiling of Human Colorectal Cancer Cell Lines with Diverse Mutations Using a High-Throughput Custom Multiplexed Analyte Panel

Cell lines that represent diverse mutational backgrounds in tumor suppressor or oncogene drivers such as TP53, KRAS, BRAF, PIK3CA, APC, TRK, CTNNB1, BRCA2, TGFB2, and PTEN were selected for the analyses reported (Table 1). The three cell lines HCT-116, HT-29, and KM12C were included because they represent varied mutational profiles and were predicted to respond differently to differing therapeutic mechanisms of action. Importantly, we included both microsatellite stable (MSS) and microsatellite instability positive (MSI+) cell lines to observe how microsatellite status impacts response to small-molecule treatment, which has implications in combination with checkpoint blockade therapies.

TABLE 1
Mutational background of selected colon cancer cell lines

| CRC Cell Line | | MSI/MSS | | | | | | |
|---|---|---|---|---|---|---|---|---|
| Name | Species | status | TP53 | HRAS | NRAS | KRAS | BRAF | PIK3CA |
| HCT-116 | human | MSI | WT | WT | WT | MT | WT | MT |
| HT-29 | human | MSS | MT | WT | WT | WT | MT | MT |
| KM12C | human | MSI | MT | WT | WT | WT | WT | WT |

| CRC Cell Line | PTEN | | | | | | |
|---|---|---|---|---|---|---|---|
| Name | expression | APC | TRK | CTNNB1 | ACVR2A | BRCA2 | TGFBR2 |
| HCT-116 | positive | WT | WT | MT | MT | MT | WT |
| HT-29 | positive | MT | WT | WT | WT | WT | MT |
| KM12C | null | MT | MT | UN | MT | MT | MT |

Abbreviation: WT: wild type; MT: mutant; UN: unknown. HCT-116, HT-29, and KM12C colon cancer cell line mutational backgrounds.

TABLE 2
Drug classes of selected small-molecule drugs

| Drug | Class |
|---|---|
| Trametinib | MEK1/2 inhibitor |
| Crizotinib | Tyrosine kinase inhibitor |
| Larotrectinib | TRK inhibitor |
| Selpercatinib | RET inhibitor |
| Vemurafenib | BRAF inhibitor |
| Regorafenib | Tyrosine kinase inhibitor |
| ONC201 | Imipridone |
| ONC206 | Imipridone |
| ONC212 | Imipridone |
| Rucaparib | PARP inhibitor |
| Olaparib | PARP inhibitor |
| Selumetinib | MEK inhibitor |
| Dabrafenib | BRAF inhibitor |
| Dasatinib | Tyrosine kinase inhibitor |
| Duvelisib | PI3K inhibitor |
| 9-ING-41 | GSK-3 inhibitor |

Drug name is listed in the first column and drug class is listed in the second column.

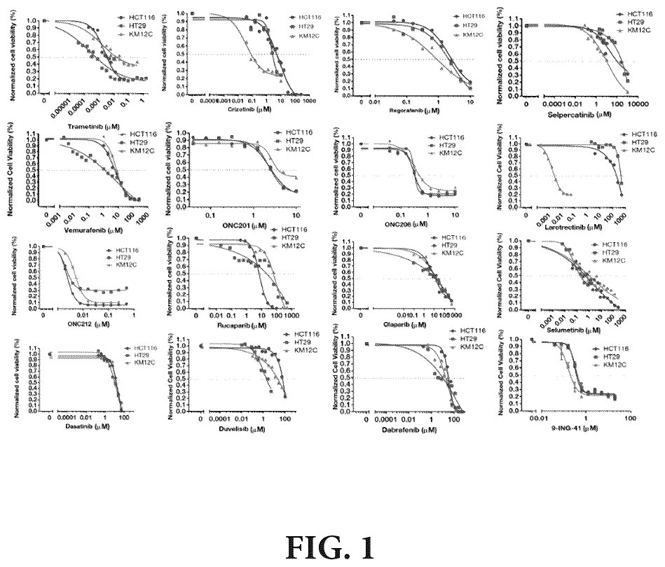

Selected oncologic small-molecules with distinct mechanisms of action encompassed several classes of drugs such as PARP-, MEK-, and BRAF-inhibitors, among others (Table 2). We primarily selected FDA-approved small-molecules, but also included several experimental small-molecules in oncology that target commonly dysregulated pathways in cancer. We were especially interested in the results of the experimental drugs that are either currently in clinical trials or are planned for clinical trials in the near future (such as GSK-3 inhibitor 9-ING-41, and Imipridones ONC201, ONC206, and ONC212). The selected cell lines displayed a range of susceptibility to the drug panel (FIG. 1).

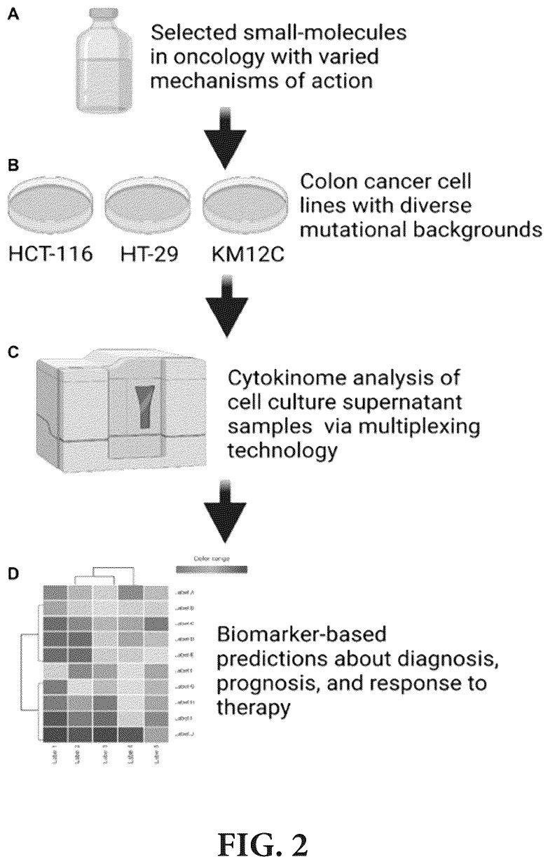

We designed a high-throughput custom multiplex cytokine, chemokine, and growth factor profiling panel based on both pro- and anti-inflammatory markers, as well as cytokines and chemokines involved in the recruitment and activation of immune cells such as natural killer (NK) and T cells. Cell lines were treated with the drug panel and cell culture supernatants were analyzed using Luminex 200 technology (FIG. 2). Cell lines were treated at differing concentrations (IC-10, IC-30, IC-50, IC-70, and IC-90) to determine dose-response effects for each small-molecule. It is important to emphasize that the panel was designed to analyze soluble factors that are secreted or shed by tumor cells post-treatment with drug.

To analyze these results, we graphed dose-response values for each drug and cell line combination and calculated the linear regression. To generate heat maps that rank analytes for each drug and cell line combination from most-downregulated to most up-regulated, we utilized the slope of the linear regression. We then examined the top five most up- and down-regulated analytes from each heat map.

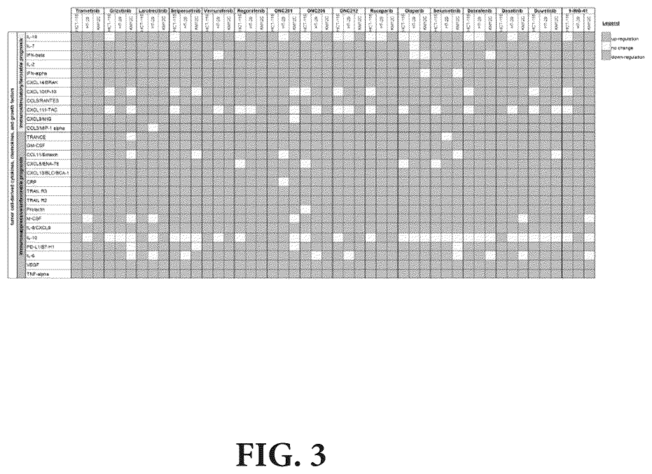

To better visualize the data, we created a summary heatmap that grouped cytokines, chemokines, and growth factors into two categories: (1) analytes that are correlated with immunosuppression or unfavorable prognosis (Tominaga T, et al., PLoS One. 2019; 14:e0212978) or (2) analytes that are correlated with immunostimulation or favorable prognosis in the context of CRC, specifically (Cao Y, et al., Front Cell Dev Biol. 2021; 9:646252) (FIG. 3). When designing these two groups, we focused on the implications of a particular analyte when colon cancer cell-derived.

Class Effects on Cytokine, Chemokine, and Growth Factor Profiles were Observed Across Multiple Human Colorectal Cancer Cell Lines

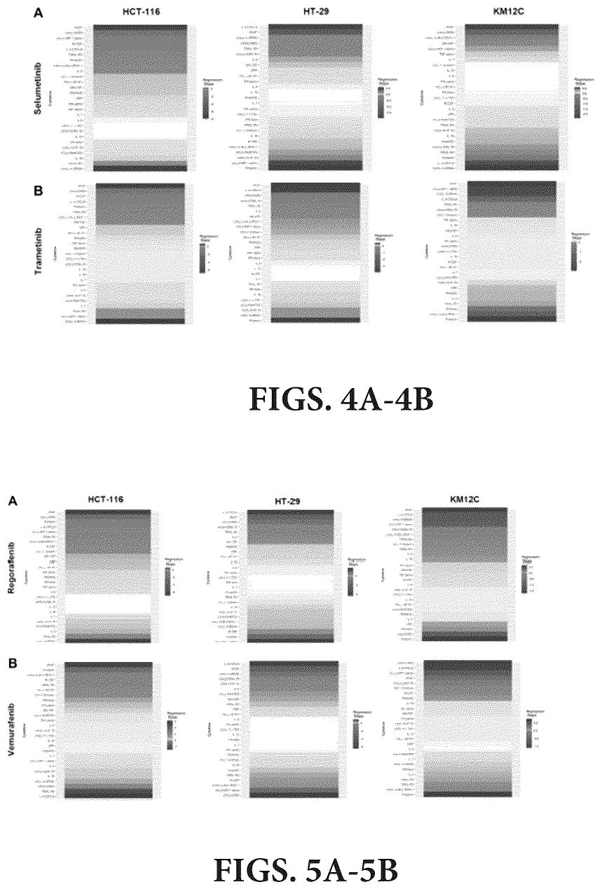

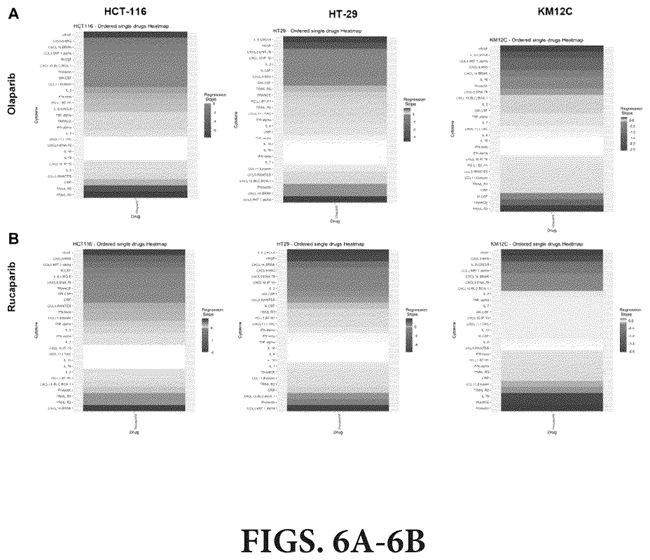

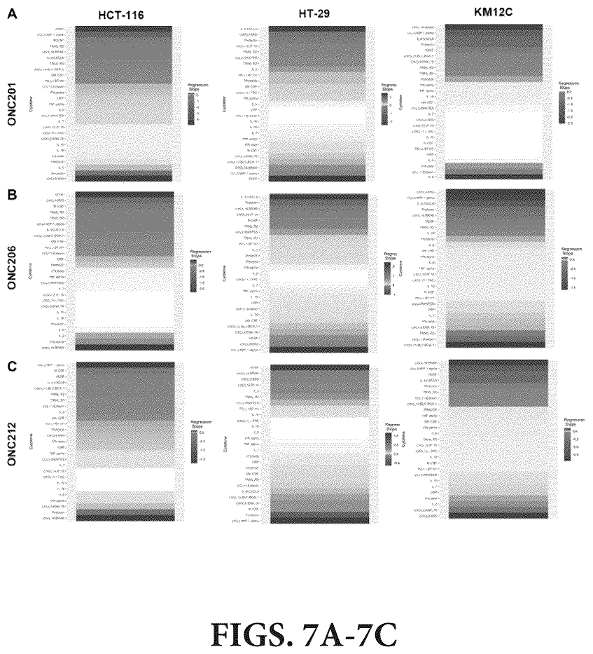

There were several classes of drugs of which we tested more than one inhibitor and observed class effects. Drug classes where we tested multiple compounds included MEK inhibitors, BRAF inhibitors, PARP inhibitors, and Imipridones. For MEK inhibitors, we tested both Trametinib and Selumetinib, and saw similarities in the top five most-downregulated analytes in response to both drugs across all three cell lines (6 groups total) (FIG. 4). We saw decreases in VEGF (6 out of 6), CXCL9/MIG (5 out of 6), and IL-8/CXCL8 (5 out of 6). The analytes that most notably increased after treatment with MEK inhibitors in all cell lines were CXCL14/BRAK (4 out of 6), Prolactin (4 out of 6), and CCL5/RANTES (4 out of 6). Next, for BRAF inhibitors Dabrafenib and Vemurafenib we again saw similar trends in response across all cell lines tested (FIG. 5). We observed decreases in VEGF (6 out of 6), and IL-8/CXCL8 (5 out of 6). By contrast, we observed increases in soluble TRAIL-R2 (sTRAIL-R2) (4 out of 6). Next, PARP inhibitors included Olaparib and Rucaparib and once again we observed notable decreases in VEGF (6 out of 6), CXCL9 (5 out of 6), IL-8 (5 out of 6), CCL3/MIP-1 alpha (4 out of 6), and CXCL14/BRAK (4 out of 6) (FIG. 6). The analytes that increased as a class effect included sTRAIL-R2 (5 out of 6), and Prolactin (4 out of 6). Lastly, for Imipridones we tested three different compounds including ONC201, ONC206, and ONC212 (9 groups) (FIG. 7). We saw notable decreases in VEGF (6 out of 9), CCL3/MIP-1 alpha (5 out of 9), CXCL14/BRAK (6 out of 9), IL-8/CXCL8 (6 out of 9), and Prolactin (5 out of 9). In contrast, we saw increases in CXCL5/ENA-78 (6 out of 9).

Drug Effects on Cytokine, Chemokine, and Growth Factor Profiles were Observed Across Multiple Cell Lines

We also evaluated several drugs that belonged to additional classes of small molecule compounds. First, we looked at GSK-3 inhibitor 9-ING-41 and saw decreases in VEGF (3 out of 3), CXCL9/MIG (3 out of 3), and CCL3/MIP-1 alpha (2 out of 3). Meanwhile, we observed increases in CXCL14 (3 out of 3), IL-8/CXCL8 (2 out of 3), sTRAIL-R2 (3 out of 3), and sTRAIL-R3 (2 out of 3). Next, we focused on Crizotinib, a c-MET inhibitor, and saw decreases in VEGF (3 out of 3), CXCL9/MIG (2 out of 3), and CXCL13/BLC/BCA-1 (2 out of 3). In contrast, we observed increases in IL-8/CXCL8 (2 out of 3), sTRAIL-R2 (2 out of 3), Prolactin (2 out of 3), and CXCL14 (2 out of 3). Next, we examined Dasatinib, a tyrosine kinase inhibitor, and observed decreases in IL-8/CXCL8 (3 out of 3), VEGF (2 out of 3), CXCL9/MIG, and CXCL5 (2 out of 3). In contrast, we noted increases in sTRAIL-R2 (3 out of 3), CXCL14/BRAK (3 out of 3), and Prolactin (2 out of 3). We next examined Duvelisib, a PI3K inhibitor and saw decreases in CCL3/MIP-1 alpha (2 out of 3), VEGF (3 out of 3), sTRAIL-R2 (2 out of 3), IL-8/CXCL8 (2 out of 3), CXCL9/MIG (2 out of 3) and CXCL14/BRAK (2 out of 3). We observed an increase in Prolactin (2 out of 3). We next analyzed Larotrectinib, a TRK inhibitor, and noted decreases in VEGF (3 out of 3), CXCL9/MIG (3 out of 3), CCL3/MIP-1 alpha (2 out of 3), and IL-8/CXCL8 (2 out of 3). Meanwhile, we observed increases in CCL5/RANTES (2 out of 3), IL-18/IL-1F4 (2 out of 3), and Prolactin (2 out of 3). We also analyzed Regorafenib, a multikinase inhibitor, and again noted decreases in VEGF (3 out of 3), CXCL9/MIG (3 out of 3), IL-8/CXCL8 (3 out of 3), CCL3 (2 out of 3), and CXCL5/ENA-78 (2 out of 3). In contrast, we noted increases in CXCL14/BRAK (2 out of 3), and Prolactin (2 out of 3). We then examined RET inhibitor Selpercatinib, and observed decreases in VEGF (3 out of 3), CXCL14/BRAK (2 out of 3), CCL3/MIP-1 alpha (2 out of 3), and sTRAIL-R2 (2 out of 3). Lastly, we saw increases in IL-8/CXCL8 (2 out of 3), CXCL5/ENA-78 (3 out of 3), and Prolactin (2 out of 3).

### Discussion

The most commonly downregulated analyte in response to all treatment conditions was vascular endothelial growth factor (VEGF). VEGF is an angiogenic factor that is upregulated in many cancer types, including CRC, and promotes tumor angiogenesis. In CRC, VEGF expression in tumor tissue and patient plasma samples correlates with disease progression and metastasis. Cao D, et al., BMC Cancer. 2009; 9:432. Moreover, VEGF-positive tumors (Bendardaf R, et al., Anticancer Res. 2008; 28:3865-70), high post-operative plasma VEGF concentrations (Werther K, et al., Br J Cancer. 2002; 86:417-23), and high serum VEGF levels are correlated with decreased overall survival in CRC. Bestas R, et al., J Clin Oncol. 2008; 26:S15070. The downregulation of VEGF that we observed as a common trend despite heterogenous cell line mutational profiles and therapeutic mechanisms of action could suggest the possibility of off-target or non-specific effects. The identification of VEGF as a tumor cell-secreted marker that is commonly altered by small molecule compounds will require further interrogation. Another commonly downregulated analyte was CXCL9/MIG, an important chemokine for both recruitment and activation of leukocytes mediated by binding to CXCR3, a receptor expressed on activated T cells. It has been shown that expression of CXCL9/MIG is higher in patients with colon cancer as compared to healthy controls. Kistner L, et al., Oncotarget. 2017; 8:89998-90012. Furthermore, CXCL9/MIG expression was corelated with the presence of tumor-infiltrating lymphocytes as well as post-operative survival. Kistner L, et al., Oncotarget. 2017; 8:89998-90012. Next, we saw recurrences in IL-8/CXCL8 down-regulation post-treatment with our drug panel. IL-8/CXCL8 expression is significantly associated with colorectal tumorigenesis and metastasis (Bie Y, et al., Dis Markers. 2019; 2019:8023460) and has been suggested as a therapeutic target for this reason. Moreover, it is known that IL-8/CXCL8 induces epithelial-mesenchymal transition (EMT) in tumor cells via the PI3K/Akt signaling axis. Shen T, et al., Oncol Rep. 2017; 37:2095-100. Another chemokine commonly downregulated was CCL3/MIP-1 alpha, which plays an important role in lymphocyte recruitment, activation, proliferation, and differentiation in colon cancer murine models. Allen F, et al., Front Immunol. 2017; 8:1390. Lastly, we observed a common post-treatment decrease in CXCL14/BRAK, a small chemokine with controversial effects in tumorigenesis. Westrich J A, et al., Mol Carcinog. 2020; 59:794-806. The clinical correlation of this biomarker with disease prognosis remains unclear at this time, as several have reported that elevated levels of CXCL14/BRAK expression in tumor sections correlates with worse overall survival (Zeng J, et al., J Transl Med. 2013; 11:6), yet others have reported the opposite (Lin K, et al., Mol Med Rep. 2014; 10:1561-68).

Interestingly, CXCL14/BRAK was also among one of the analytes most commonly upregulated. We also noted a common increase in the hormone prolactin, which is commonly overexpressed in patients with colorectal cancer [Soroush A R, et al., BMC Cancer. 2004; 4:97]. Next, we observed a recurrent upregulation of CCL5/RANTES which is chemotactic for many leukocytes and plays an important role in immune cell recruitment to inflammatory sites. In contrast, the CCR5/CCL5 axis has also been reported to play a role in the proliferation, metastasis, and formation of an immunosuppressive microenvironment. Aldinucci D, et al., Mediators Inflamm. 2014; 2014:292376. Tumor-derived CCL5/RANTES has been shown to enhance regulatory T cell-mediated killing of cytotoxic T cells in colon cancer. Chang L Y, et al., Cancer Res. 2012; 72:1092-102. Moreover, CCL-5 deficiency has been shown to increase tumor infiltrating CD8+ T cells in the context of CRC. Zhang S, et al., Cell Death Dis. 2018; 9:766. Lastly, we noted an upregulation of CXCL5/ENA-78 under many of the treatment conditions across several cell lines, which may induce colorectal cancer angiogenesis. Chen C, et al., Cell Death Dis. 2019; 10:178.

We also monitored soluble receptors TNF-related apoptosis-inducing ligand Receptor 2 (sTRAIL-R2)/Death Receptor 5 (sDR5) and TNF-related apoptosis-inducing ligand Receptor 3 (sTRAIL-R3). TRAIL-R2 is well-known as a cell surface receptor that triggers apoptosis upon binding with its cognate ligand, TNF-related apoptosis-inducing ligand (TRAIL). In contrast, TRAIL-R3 is known as a decoy receptor for TRAIL, as it lacks a cytoplasmic death domain rendering it unable to induce apoptosis. The soluble versions of these receptors presumably both function as decoy receptors that can bind and prevent TRAIL-mediated apoptosis. To our knowledge, soluble TRAIL receptors have not yet been characterized as potential biomarkers of immune response to therapeutics in the context of cancer. These may be novel biomarkers for assessing the innate immune system as impacted by cancer therapeutics and would be especially relevant in the context of immunotherapies such as αPD-1, αPD-L1, and αCTLA4. Furthermore, these are relevant biomarkers in the context of TRAIL-receptor agonists such as ABBV-621, IGN-8444, INBRX-109, and AMG-655 which could be bound by sTRAIL-R2, potentially reducing therapeutic efficacy. However, the extent to which soluble TRAIL-R2/R3 can predict therapeutic efficacy in humans or in mice remains to be determined.

We also analyzed soluble receptor ligand programed-death ligand 1 (sPD-L1). PD-L1 is a transmembrane molecule that belongs to the B7 family and acts by binding to PD-1 on the surface of lymphocytes to inhibit the differentiation and proliferation of immune cells. Shan T, et al., Int J Clin Exp Pathol. 2019; 12:1764-69. In CRC, elevated expression of PD-L1 is associated with poor prognosis, survival, and lymph node metastasis. Furthermore, PD-L1 expression due to IFN-γ signaling predicts poor survival in CRC. Zhao T, et al., Oncol Lett. 2020; 20:1127-34. PD-L1 is thought to be most relevant as a biomarker in the context of immunotherapy, where many have described both predictive and prognostic roles of PD-L1 in colorectal cancer, and several other cancer types. Li Y, et al., Front Pharmacol. 2019; 10:139. PD-L1 is currently being evaluated as a biomarker of poor prognosis in patients with CRC undergoing immunotherapy. The soluble version of PD-L1, specifically, is an emerging biomarker of focus in CRC and increased sPD-L1 expression post-neoadjuvant chemoradiotherapy is correlated with worse disease-free survival. Tominaga T, et al., PLoS One. 2019; 14:e0212978.

We observed heterogeneity in cytokine, chemokine, and growth factor responses across cell lines and across drug treatments. We present our results as a novel platform with a large panel of relevant cytokines, chemokines, and growth factors that can impact therapeutic and immune response in the complex tumor microenvironment.

### Materials and Methods

### Cell Culture

Human colorectal cancer cells HCT-116, HT-29, and KM12C were used in this study. HCT-116 and HT-29 were cultured in McCoy's 5A (modified) Medium supplemented with 10% FBS and 1% Penicillin-Streptomycin. KM12C cells were cultured in Eagle's Minimal Essential Medium Supplemented with 10% FBS and 1% Penicillin-Streptomycin. All cell lines were incubated at 37° C. in a humidified atmosphere containing 5% CO₂. Cell lines were authenticated and tested to ensure the cultures were free of mycoplasma infection.

### Measurement of Cell Viability

Cells were plated at a density of 3×10³ cells per well in a 96-well plate (Greiner Bio-One, Monroe, NC, USA). Cell viability was assessed using the CellTiter Glo assay (Promega, Madison, WI, USA). Cells were mixed with 25 μl of CellTiter-Glo reagents in 100 μl of culture volume, and bioluminescence imaging was measured using the Xenogen IVIS imager (Caliper Life Sciences, Waltham, MA, USA).

### Collection of Culture Supernatants Used in Cytokine Measurements

Cells were plated at 3.5×10⁴ cells in a 48 well plate (Thermo Fisher Scientific, Waltham, MA, USA) in complete medium and incubated at 37° C. with 5% CO₂. At 24 hours after plating, almost all the tumor cells were adherent to the bottom of the flask and the complete medium was replaced with drug-containing medium. Subsequently, the culture supernatants were collected after 48 hours of incubation and were frozen at −80° C. until the measurement of cytokines was performed. The day of analysis, samples were thawed and centrifuged to remove cellular debris.

### Cytokine, Chemokine, and Growth Factor Profiling

An R&D systems Human Premixed Multi-Analyte Kit (R&D Systems, Inc., Minneapolis, MN) was run on a Luminex 200 Instrument (LX200-XPON-RUO, Luminex Corporation, Austin, TX) according to the manufacturer's instructions. Cell culture supernatant levels of TNF-alpha, IL-6, IL-8/CXCL8, Ferritin, IFN-beta, IL-10, CCL2/JE/MCP-1, VEGF, CXCL13/BLC/BCA-1, IFN-gamma, CCL20/MIP-3 alpha, CCL3/MIP-1 alpha, CCL22/MDC, CCL4/MIP-1 beta, IL-4, IL-17/IL-17a, TRAIL R2/TNFRSF10B, GM-CSF, CXCL5/ENA-78, CXCL9/MIG, G-CSF, CXCL11/I-TAC, Granzyme B, CCL5/RANTES, Prolactin, IFN-alpha, CXCL14/BRAK, IL-12/IL-23 p40, CXCL10/IP-10/CRG2, CCL7/MCP-3/MARC, IL-7, CCL8/MCP-2, TRANCE/TNFSF11/RANK L, IL-15, TRAIL R3/TNFRSF10C, CCL11/Eotaxin, IL-18/IL-1F4, TRAIL/TNFSF10, IL-21, and C-Reactive Protein/CRP were measured.

### Bioinformatics Analysis

A quantitative analysis with 6 standards and a minimum of 50 counts per bead region was used with the Luminex to generate analyte values reported as picograms/milliliter (pg/mL). Cytokine concentrations less than the lower limit of detection for each particular cytokine were recoded as zero. Cytokines without detectable expression levels were removed from further analysis of each cell line. Each drug was tested at different inhibitory concentrations (IC-10, IC-30, IC-50, IC-70, and IC-90), and these varying concentrations were used to measure a dose-response effect on cytokine expression. Cytokine dose-response effect was modeled by simple linear regression for each drug. The slopes of the linear regressions were compared. Data analysis and visualization were generated using R (R Development Core Team, 2020).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating a subject having colon cancer with a small molecule drug for treating colon cancer, comprising
determining the effect of the small molecule drug on the release of a plurality of immunomodulating factors by colon cancer cells; and
treating the subject with a therapeutically effect amount of the drug if the effect of the drug on the release of immunomodulating factors indicates that the drug promotes anti-tumor immunity, or
selecting a different drug for treating colon cancer if the effect of the drug on immunomodulating factor release indicates that the drug does not promote anti-tumor immunity.

2. The method of claim 1, wherein the immunomodulating factors comprise cytokines.

3. The method of claim 2, wherein the cytokines are selected from the group consisting of

4. The method of claim 1, wherein the immunomodulating factors comprise chemokines.

5. The method of claim 4, wherein the chemokines are selected from the group consisting of

6. The method of claim 1, wherein the immunomodulating factors comprise growth factors.

7. The method of claim 6, wherein the growth factors are selected from the group consisting of

8. The method of claim 1, wherein the small molecule drug for treating colon cancer is a tyrosine kinase inhibitor.

9. The method of claim 1, wherein the small molecule drug for treating colon cancer is an imipridone compound.

10. The method of claim 1, wherein the small molecule drug for treating colon cancer is selected from the group consisting of MEK inhibitors, RET inhibitors, BRAF inhibitors, PARP inhibitors, P13K inhibitors, and GSK-3 inhibitors.

11. The method of claim 1, wherein the effect of the small molecule drug on the release of a plurality of immunomodulating factors is evaluated in a plurality of different types of colon cancer cells.

12. The method of claim 1, wherein the levels of the immunomodulating factors are determined using an immunoassay.

13. The method of claim 12, wherein the levels of the immunomodulating factors are determined using a multiplex kit.

14. The method of claim 1, wherein the subject is a human subject.

15. A method of evaluating treatment a subject having colon cancer being treated with a therapeutically effective amount of a small molecule drug for treating colon cancer, comprising
determining the effect of the small molecule drug on the release of a plurality of immunomodulating factors by colon cancer cells; and
continuing to treat the subject if the effect of the drug on the release of immunomodulating factors indicates that the drug promotes anti-tumor immunity, or
changing treatment to a different drug for treating colon cancer if the effect of the drug on immunomodulating factor release indicates that the drug does not promote anti-tumor immunity.

16. The method of claim 15, wherein the immunomodulating factors comprise cytokines.

17. The method of claim 15, wherein the immunomodulating factors comprise chemokines.

18. The method of claim 15, wherein the immunomodulating factors comprise growth factors.

19. The method of claim 15, wherein the effect of the small molecule drug on the release of a plurality of immunomodulating factors is evaluated in a plurality of different types of colon cancer cells.

20. The method of claim 15, wherein the levels of the immunomodulating factors are determined using an immunoassay.

21. The method of claim 15, wherein the levels of the immunomodulating factors are determined using a multiplex kit.
